(19) United States
(12) Patent Application Publication
(10) Pub. No.: US 2022/0415444 A1
(43) Pub. Date: Dec. 29, 2022

(54) ACCELERATING NUCLEIC ACID SEQUENCING DATA WORKFLOWS USING A RAPID COMPUTATION OF HAMMING DISTANCE

(71) Applicant: University of Washington, Seattle, WA (US)

(72) Inventors: Ling-Hong Hung, Seattle, WA (US); Ka Yee Yeung-Rhee, Seattle, WA (US)

(21) Appl. No.: 17/850,739

(22) Filed: Jun. 27, 2022

Related U.S. Application Data
(60) Provisional application No. 63/216,464, filed on Jun. 29, 2021.

(51) Int. Cl. G16B 30/10

(52) U.S. Cl. CPC G16B 30/10

(57) ABSTRACT

In some embodiments, a computer-implemented method of comparing strings representing nucleotide sequences is provided. A plurality of values representing Hamming distances between first strings and second strings are determined by converting value characters in the first string and the second string to a one hot encoding and converting any unknown characters in the first string and the second string to a zero value to create a first bit representation and a second bit representation; comparing the first bit representation and the second bit representation using a bitwise XOR operation to obtain a bitwise XOR result; counting a number of bits in the bitwise XOR result and multiplying the bitwise XOR result by two to obtain a bitcount result; and adjusting the bitcount result based on unknown characters in at least one of the first string and the second string to obtain the value representing the Hamming distance.

### CROSS REFERENCE TO RELATED APPLICATION

This application claims the benefit of Provisional Application No. 63/216,464, filed Jun. 29, 2021, the entire disclosure of which is hereby incorporated by reference herein for all purposes.

### STATEMENT OF GOVERNMENT SUPPORT

This invention was made with government support under Grant No. RO1 GM126019, awarded by the National Institutes of Health. The government has certain rights in the invention.

### BACKGROUND

Massive amounts of high-throughput sequencing data have been generated in recent years. As an example, the Sequence Read Archive (SRA) database, the largest available public repository of high-throughput sequencing data hosted by the National Center for Biotechnology Information (NCBI), consists of over 51 quadrillion total bases. New long-read sequencing technologies, such as Pacific Biosciences SMRT and Oxford Nanopore MinION, have emerged. Most of the sequencing data currently available from public repositories are generated using short read sequencing technologies such as Illumina, which has ˜75% global market share in the genetic sequencing industry and generates read lengths in the range of 100-300 base pairs (bp). In contrast, Pacific Biosciences SMRT and Oxford Nanopore MinION generate read lengths in the range of 10-100 kb, and up to 1000 kb respectively. These long-read sequencing technologies have great potential in de novo genome assembly, identification of transcript isoforms, structural variant detection, disease diagnosis and prognosis.

Sequencing data are typically represented as character strings. For example, DNA sequences are represented as character strings from the alphabet A, C, G or T. Aligning two or more sequences can be used to identify regions of similarity that may indicate functional, structural, or evolutionary relationships between the sequences. When analyzing high-throughput sequencing data, a computational bottleneck is read alignment in which reads (sequences) are mapped to a common reference genome or transcriptome. Therefore, a common basic operation is the comparison of two character strings to determine an amount of similarity between the two character strings.

The Hamming distance is a metric that is often used to represent similarity between sequences. The Hamming distance between two character strings of equal length is the number of positions at which the corresponding characters are different. The Hamming distance is often used to compare two bitstrings in many applications, including sequence alignment but also other applications such as in error-correcting codes, information retrieval, and web search engines.

Suppose the length of the two character strings is n. A straight forward implementation of comparing each entry in the two character strings to determine the Hamming distance would lead to a linear time O(n) algorithm. Because so many comparisons need to be conducted while aligning read sequences to a reference genome, using an O(n) algorithm for computation of Hamming distances is a serious bottleneck in alignment processing, particularly when the length of individual read sequences increases. What is desired is a more efficient technique for quantifying similarities between character strings so that nucleic acid analysis workflows can be accelerated.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In some embodiments, a computer-implemented method of comparing a first set of nucleotide sequences represented by a set of first strings to a second set of nucleotide sequences represented by a set of second strings is provided. A computing device receives the set of first strings representing the nucleotide sequences of the first set of nucleotide sequences. The computing device determines a plurality of values representing Hamming distances between the first strings and the second strings representing the second set of nucleotide sequences by performing actions comprising, for each pairing of a first string and a second string: converting any value characters in the first string and the second string to a one hot encoding and converting any unknown characters in the first string and the second string to a zero value to create a first bit representation and a second bit representation, wherein each value character represents a nucleotide in a plurality of nucleotides; comparing the first bit representation and the second bit representation using a bitwise XOR operation to obtain a bitwise XOR result; counting a number of bits in the bitwise XOR result and multiplying the bitwise XOR result by two to obtain a bitcount result; and adjusting the bitcount result based on unknown characters in at least one of the first string and the second string to obtain the value representing the Hamming distance. The computing device provides a result based on the determined plurality of values.

In some embodiments, a non-transitory computer-readable medium having computer-executable instructions stored thereon is provided. The instructions, in response to execution by one or more processors of a computing device, cause the computing device to perform actions for comparing a first set of nucleotide sequences represented by a set of first strings to a second set of nucleotide sequences represented by a set of second strings, the actions comprising: receiving, by the computing device, the set of first strings representing the nucleotide sequences of the first set of nucleotide sequences; determining, by the computing device, a plurality of values representing Hamming distances between the first strings and the second strings representing the second set of nucleotide sequences by performing actions comprising, for each pairing of a first string and a second string: converting any value characters in the first string and the second string to a one hot encoding and converting any unknown characters in the first string and the second string to a zero value to create a first bit representation and a second bit representation, wherein each value character represents a nucleotide in a plurality of nucleotides; comparing the first bit representation and the second bit representation using a bitwise XOR operation to obtain a bitwise XOR result; counting a number of bits in the bitwise XOR result and multiplying the bitwise XOR result by two to obtain a bitcount result; and adjusting the bitcount result based on unknown characters in at least one of the first string and the second string to obtain the value representing the Hamming distance; and providing a result based on the determined plurality of values.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a block diagram that illustrates a non-limiting example embodiment of a system for processing sequence information according to various aspects of the present disclosure.

FIG. 2 is a block diagram that illustrates aspects of a non-limiting example embodiment of a sequence comparison computing device according to various aspects of the present disclosure.

FIG. 3 is a flowchart that illustrates a non-limiting example embodiment of a method of aligning sequence reads to a reference genome according to various aspects of the present disclosure.

FIG. 4 is a flowchart that illustrates a non-limiting example embodiment of a method of clustering sequence reads according to various aspects of the present disclosure.

FIG. 5 is a flowchart that illustrates a non-limiting example embodiment of a procedure for determining a Hamming distance between a first sequence and a second sequence according to various aspects of the present disclosure.

FIG. 6A illustrates a non-limiting example embodiment of a one hot encoding according to various aspects of the present disclosure.

FIG. 6B illustrates the number of one bits that are present when any two characters are compared using an exclusive or (XOR, or ^) operation.

FIG. 6C illustrates the pairwise distance that is assigned between the characters by virtue of the one hot encoding.

### DETAILED DESCRIPTION

The present disclosure describes techniques for accelerating the computation of the Hamming distance between two strings (including but not limited to strings representing nucleic acid sequences) by encoding each sequence as a bitstring. Bitwise operations are used to generate new comparison strings. Counting the bits in these strings gives us the Hamming distance between the two sequences. This allows for characters in a sequence to be compared simultaneously using a small number of fast, low-level operations. On commodity-level general purpose CPUs that are currently widely available, the word length is usually 64 bits. On such a processor, the bitwise operations described herein allow for 16 comparisons to be made simultaneously because 4 bits are used to encode each nucleotide. Using vector units which allow for 128-, 256-, or 512-bit operations, up to 128 comparisons can be made simultaneously. Specialized CPUs, GPUs, FPGAs, and/or ASICs that support even wider bit operations can allow for even more simultaneous comparisons.

The present disclosure relates to encoding character strings such as a nucleic acid sequences using a bit representation, thus reducing the problem of computing the Hamming distance between two character strings into bitwise operations. Specifically, it is shown that the Hamming distance can be derived from performing bitwise exclusive or (XOR, or ^), bitwise and (AND, or &), and bit counting operations. The present disclosure also describes the result of empirical experiments that demonstrate substantial computational speedup, and identifies relevant applications in supporting bioinformatic workflows.

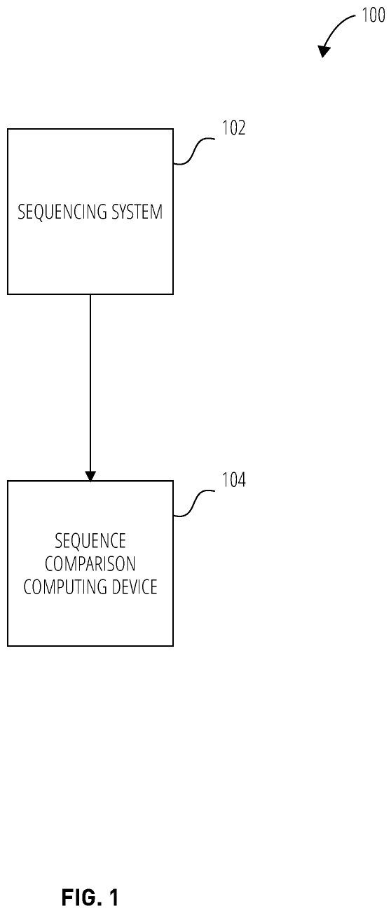

FIG. 1 is a block diagram that illustrates a non-limiting example embodiment of a system for processing sequence information according to various aspects of the present disclosure. In the system 100, a sequencing system 102 provides sequence read information to a sequence comparison computing device 104 via any suitable communication technology, including but not limited to wired technologies (e.g., Ethernet, USB, Firewire, etc.), wireless technologies (e.g., Bluetooth, Wi-Fi, 5G, etc.), and by exchanging removable computer-readable media (e.g., flash memory, optical discs, etc.).

The sequencing system 102 may be any type of system that generates sequencing information. One non-limiting example of a sequencing system 102 is a MinION system from Oxford Nanopore Technologies. Another non-limiting example of a sequencing system 102 is a Sequel system from Pacific Biosciences. In some embodiments, the sequencing system 102 provides sequence read information to the sequence comparison computing device 104 in real time as the sequencing system 102 is sequencing one or more samples. In some embodiments, the sequencing system 102 may generate sequence read information for a sample, store the sequence read information, and provide the sequence read information to the sequence comparison computing device 104 once sequencing is complete.

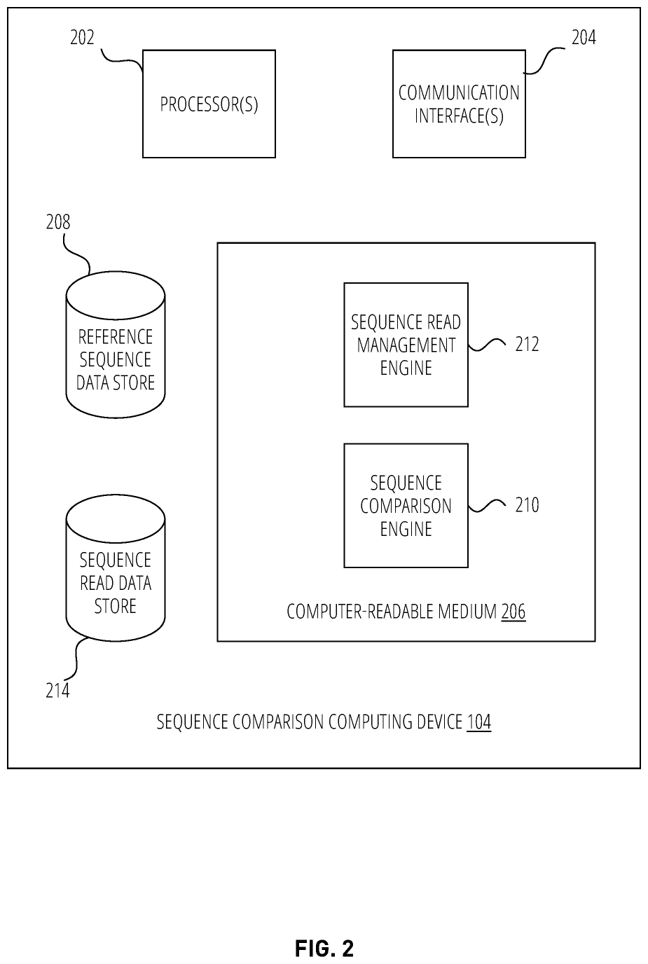

FIG. 2 is a block diagram that illustrates aspects of a non-limiting example embodiment of a sequence comparison computing device according to various aspects of the present disclosure. The illustrated sequence comparison computing device 104 may be implemented by any computing device or collection of computing devices, including but not limited to a desktop computing device, a laptop computing device, a mobile computing device, a server computing device, a computing device of a cloud computing system, and/or combinations thereof. The sequence comparison computing device 104 is configured to process sequence read information in an accelerated manner using the bitwise encoding techniques described herein.

As shown, the sequence comparison computing device 104 includes one or more processors 202, one or more communication interfaces 204, a reference sequence data store 208, a sequence read data store 214, and a computer-readable medium 206.

In some embodiments, the processors 202 may include any suitable type of general-purpose computer processor. In some embodiments, the processors 202 may include one or more special-purpose computer processors or AI accelerators optimized for specific computing tasks, including but not limited to graphical processing units (GPUs), vision processing units (VPTs), and tensor processing units (TPUs). In some embodiments, the processors 202 may include FPGAs, ASICs, or other special-purpose processors or other circuitry configured to perform the bitwise operations described herein with a wider bit depth than available in commercially available processors.

In some embodiments, the communication interfaces 204 include one or more hardware and or software interfaces suitable for providing communication links between components. The communication interfaces 204 may support one or more wired communication technologies (including but not limited to Ethernet, FireWire, and USB), one or more wireless communication technologies (including but not limited to Wi-Fi, WiMAX, Bluetooth, 2G, 3G, 4G, 5G, and LTE), and/or combinations thereof, for communicating with the sequencing system 102 and/or other computing devices.

As shown, the computer-readable medium 206 has stored thereon logic that, in response to execution by the one or more processors 202, cause the sequence comparison computing device 104 to provide a sequence read management engine 212 and a sequence comparison engine 210.

As used herein, “computer-readable medium” refers to a removable or nonremovable device that implements any technology capable of storing information in a volatile or non-volatile manner to be read by a processor of a computing device, including but not limited to: a hard drive; a flash memory; a solid state drive; random-access memory (RAM); read-only memory (ROM); a CD-ROM, a DVD, or other disk storage; a magnetic cassette; a magnetic tape; and a magnetic disk storage.

In some embodiments, the sequence read management engine 212 is configured to receive sequence reads from the sequencing system 102 and store the sequence read information in the sequence read data store 214. In some embodiments, the sequence comparison engine 210 is configured to use the bitwise operations described herein to compare sequences, including comparing sequence reads from the sequence read data store 214 to references sequences in the reference sequence data store 208, and comparing sequence reads from the sequence read data store 214 to each other.

Further description of the configuration of each of these components is provided below.

As used herein, “engine” refers to logic embodied in hardware or software instructions, which can be written in one or more programming languages, including but not limited to C, C++, C#, COBOL, JAVA™, PHP, Perl, HTML, CSS, JavaScript, VBScript, ASPX, Go, and Python. An engine may be compiled into executable programs or written in interpreted programming languages. Software engines may be callable from other engines or from themselves. Generally, the engines described herein refer to logical modules that can be merged with other engines, or can be divided into sub-engines. The engines can be implemented by logic stored in any type of computer-readable medium or computer storage device and be stored on and executed by one or more general purpose computers, thus creating a special purpose computer configured to provide the engine or the functionality thereof. The engines can be implemented by logic programmed into an application-specific integrated circuit (ASIC), a field-programmable gate array (FPGA), or another hardware device.

As used herein, “data store” refers to any suitable device configured to store data for access by a computing device. One example of a data store is a highly reliable, high-speed relational database management system (DBMS) executing on one or more computing devices and accessible over a high-speed network. Another example of a data store is a key-value store. However, any other suitable storage technique and/or device capable of quickly and reliably providing the stored data in response to queries may be used, and the computing device may be accessible locally instead of over a network, or may be provided as a cloud-based service. A data store may also include data stored in an organized manner on a computer-readable storage medium, such as a hard disk drive, a flash memory, RAM, ROM, or any other type of computer-readable storage medium. One of ordinary skill in the art will recognize that separate data stores described herein may be combined into a single data store, and/or a single data store described herein may be separated into multiple data stores, without departing from the scope of the present disclosure.

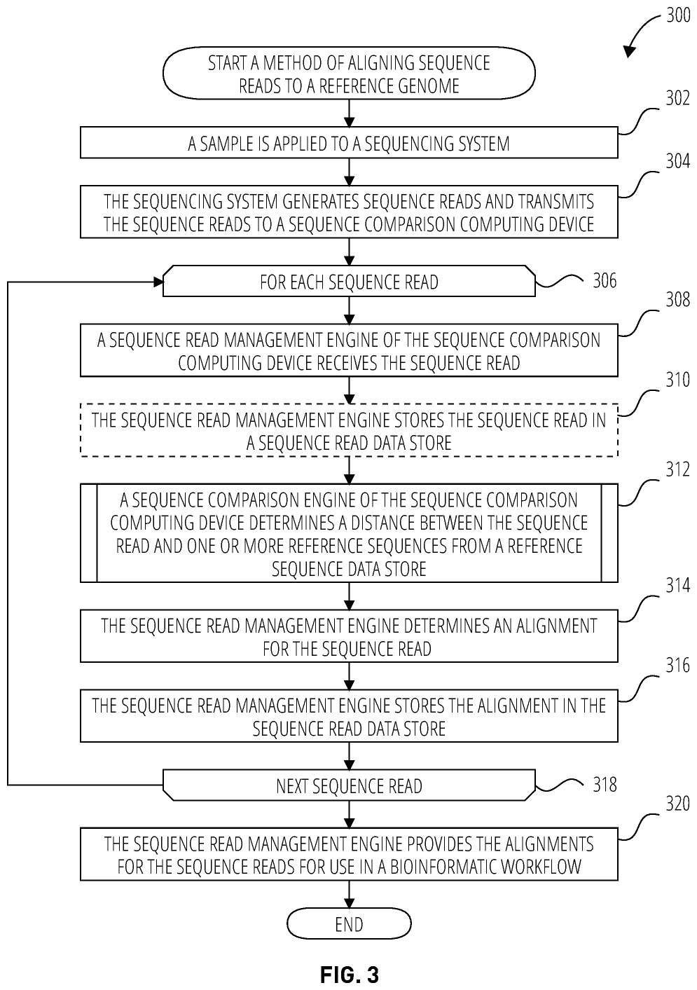

FIG. 3 is a flowchart that illustrates a non-limiting example embodiment of a method of aligning sequence reads to a reference genome according to various aspects of the present disclosure. In general, the overall method 300 is a common workflow used to align sequence reads to a reference genome, but the common workflow is enhanced by using the accelerated techniques for computing Hamming distance of the present disclosure.

From a start block, the method 300 proceeds to block 302, where a sample is applied to a sequencing system 102. The sample may be from a subject, from a biopsy, from a cell culture, or from any other suitable source. Any suitable technique associated with the sequencing system 102 may be used to apply the sample to the sequencing system 102, including combining the sample with a reagent and providing the combination to an input of the sequencing system 102.

At block 304, the sequencing system 102 generates sequence reads and transmits the sequence reads to a sequence comparison computing device 104. Each sequence read may be a character string that includes characters indicating each base that was detected, including characters for positions where bases could not be determined.

The method 300 then proceeds to a for-loop defined between a for-loop start block 306 and a for-loop end block 318, wherein each of the sequence reads is processed. From the for-loop start block 306, the method 300 proceeds to block 308, where a sequence read management engine 212 of the sequence comparison computing device 104 receives the sequence read. In some embodiments, the sequence read management engine 212 may receive the sequence read in real-time while the sequencing system 102 is sequencing a molecule and generating the sequence read. In some embodiments, the sequence read management engine 212 may receive the sequence read for a given molecule once the sequencing system 102 has completed sequencing the given molecule. In some embodiments, the sequence read management engine 212 may receive all of the sequence reads in a batch once the processing of the sample is completed.

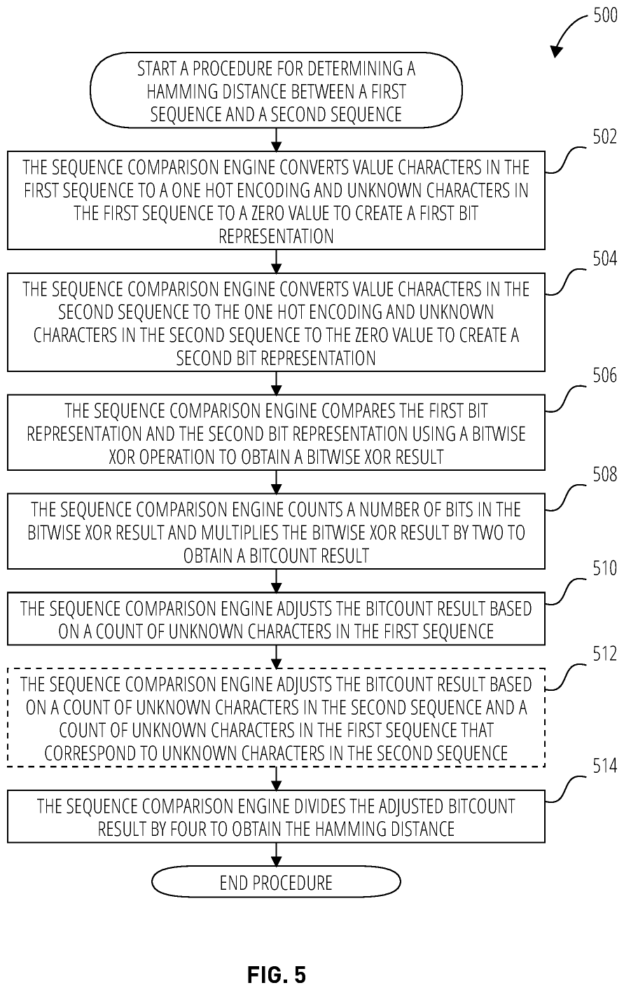

At optional block 310, the sequence read management engine 212 stores the sequence read in a sequence read data store 214. In some embodiments, the sequence read management engine 212 may store the character string received from the sequencing system 102 in the sequence read data store 214. In some embodiments, the sequence read management engine 212 may convert the sequence read to a bit representation as illustrated in FIG. 5 and may store the bit representation instead of (or in addition to) the character string in the sequence read data store 214. The actions of optional block 310 are described as optional because, in some embodiments, the sequence read management engine 212 may process the sequence read without first storing it in the sequence read data store 214.

At subroutine block 312, a sequence comparison engine 210 of the sequence comparison computing device 104 determines a distance between the sequence read and one or more reference sequences from a reference sequence data store 208. The reference sequences may include one or more portions of a human genome, a genome of a pathogen, a genetic signature of a cancer or other disease, or any other type of reference sequence. In some embodiments, the reference sequences may be or may include a sequence of interest from a reference genome; a sequence of interest from a viral insertion; a sequence of a predicted protein that is not associated with a position in the reference genome; a sequence of a predicted gene that is not associated with a position in the reference genome; and partial or complete sequences of fusion genes as a result of translocation, inversion, and deletion.

Typical sequencing workflows include a step of determining a distance between the sequence read and one or more reference sequences in order to find locations in the reference sequences that are likely to match the sequence read. In the method 300, subroutine block 312 uses an improved technique to determine the distance as illustrated in FIG. 5. Since the determination at subroutine block 312 is performed for each sequence read and for many reference sequences from the reference sequence data store 208, improvements in the speed of the determination of the distance (such as those provided by the procedure illustrated in FIG. 5) serve to greatly reduce the overall time used by the method 300.

At block 314, the sequence read management engine 212 determines an alignment for the sequence read, and at block 316, the sequence read management engine 212 stores the alignment in the sequence read data store 214. In some embodiments, the alignment may be determined based on one or more reference sequences that are found to have a low distance from the sequence read (e.g., reference sequences that are the most similar to the sequence read). The alignment indicates which reference sequence the sequence read is most likely to represent, and is thereby correlated to a particular portion of a reference genome.

The method 300 then proceeds to the for-loop end block 318. If further sequence reads remain to be processed, then the method 300 returns to for-loop start block 306 to process the next sequence read. Otherwise, if all of the sequence reads have been processed, then the method 300 advances to block 320.

At block 320, the sequence read management engine 212 provides the alignments for the sequence reads for use in a bioinformatic workflow. In some embodiments, the bioinformatic workflow may include mapping the alignments to a diagnostic panel of genes in targeted sequencing, which may be useful in diagnosing cancer, identifying a pathogen, or other uses.

Though the method 300 illustrates the alignments as being used in a bioinformatic workflow after the for-loop, in some embodiments, the alignments may be used before the for-loop is completed. For example, some sequencing systems 102 (such as the MinION system) provide functionality that allows a molecule to be ejected if it is determined that it is not a molecule of interest. In such embodiments, if the alignment indicates that a sequence read received in real-time while it is being sequenced by the sequencing system 102 is not a molecule of interest, the alignment may be used to instruct the sequencing system 102 to eject the molecule instead of sequencing it further.

The method 300 then proceeds to an end block and terminates.

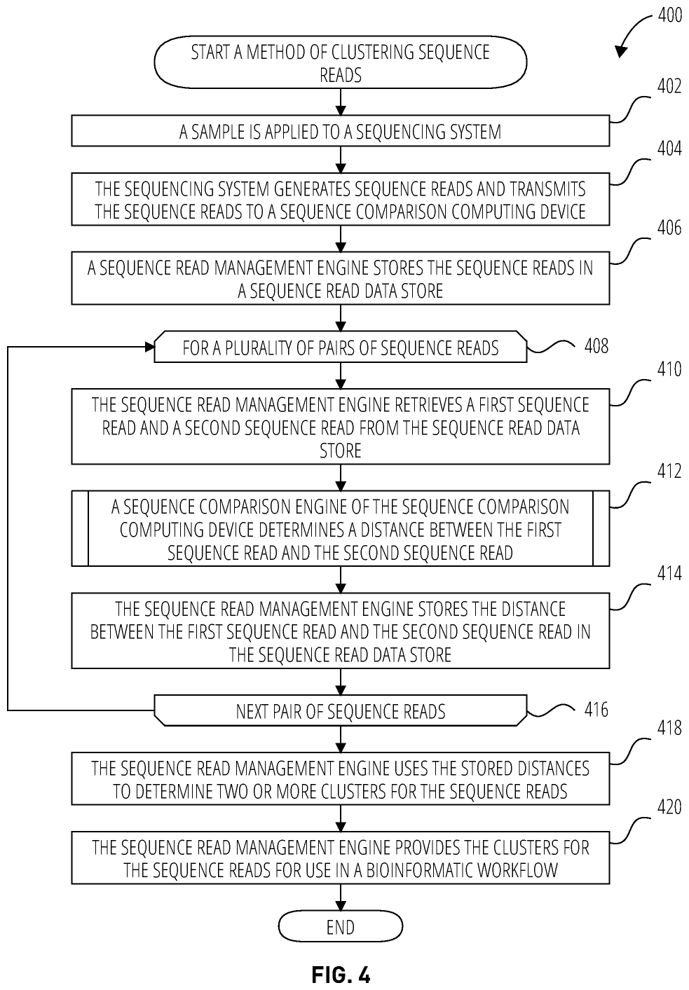

FIG. 4 is a flowchart that illustrates a non-limiting example embodiment of a method of clustering sequence reads according to various aspects of the present disclosure. In general, the overall method 400 is a common workflow used to analyze sequence reads, but the common workflow is enhanced by using the accelerated techniques for computing Hamming distance of the present disclosure.

From a start block, the method 400 proceeds to block 402, where a sample is applied to a sequencing system 102, and block 404, the sequencing system 102 generates sequence reads and transmits the sequence reads to a sequence comparison computing device 104. These actions are the same as those illustrated in block 302 and block 304 in FIG. 3, and so are not described again in detail for the sake of brevity.

At block 406, a sequence read management engine 212 stores the sequence reads in a sequence read data store 214. The method 400 then proceeds to a for-loop defined between for-loop start block 408 and for-loop end block 416, wherein the sequence reads stored in the sequence read data store 214 are compared in pairs to determine mutual distances between the sequence reads.

From for-loop start block 408, the method 400 proceeds to block 410, where the sequence read management engine 212 retrieves a first sequence read and a second sequence read (the pair of sequence reads) from the sequence read data store 214. At subroutine block 412, a sequence comparison engine 210 of the sequence comparison computing device 104 determines a distance between the first sequence read and the second sequence read. Sequence read clustering analyses typically include determining distances between pairs of sequence reads. In the method 400, subroutine block 412 uses the improved techniques disclosed herein to determine the distance as illustrated in FIG. 5. Again, since the determination at subroutine block 312 is performed at least for each pair of sequences in the sequence read data store 214, improvements in the speed of the determination of the distance (such as those provided by the procedure illustrated in FIG. 5) serve to greatly reduce the overall time used by the method 300.

At block 414, the sequence read management engine 212 stores the distance between the first sequence read and the second sequence read in the sequence read data store 214. The method 400 then proceeds to the for-loop end block 416. If further pairs of sequence reads remain to be processed, then the method 400 returns to the for-loop start block 408 to process the next pair of sequence reads. Otherwise, if all of the pairs of sequence reads have been processed, then the method 400 proceeds to block 418.

At block 418, the sequence read management engine 212 uses the stored distances to determine two or more clusters for the sequence reads. The distances indicate which pairs of sequence reads are more similar to each other (e.g., have smaller distances), and likely belong to the same cluster. At block 420, the sequence read management engine 212 provides the clusters for the sequence reads for use in a bioinformatic workflow. The cluster information may be used in any suitable bioinformatic workflow, including but not limited to finding sequence reads that are associated with the same gene, identifying protein families, and structural genomic analysis.

The method 400 then proceeds to an end block and terminates.

FIG. 5 is a flowchart that illustrates a non-limiting example embodiment of a procedure for determining a Hamming distance between a first sequence and a second sequence according to various aspects of the present disclosure. The procedure 500 uses bitwise operations to compare multiple characters from the sequences at once, and thereby greatly accelerate the computation of the Hamming distance. As stated above, in some embodiments, the first sequence and the second sequence may be sequence reads obtained from a sequencing system 102. In some embodiments, the first sequence may be a sequence read and the second sequence may be a reference sequence. In some embodiments, the first sequence and second sequence may be character strings other than character strings that represent sequence data. For the sake of simplicity, it is assumed in procedure 500 that the first sequence and the second sequence are of the same length, though one of ordinary skill in the art will recognize that sequences of different lengths could also be compared (e.g., by truncating the longer sequence or by padding the shorter sequence with appropriate characters).

From a start block, the procedure 500 proceeds to block 502, where the sequence comparison engine 210 converts value characters in the first sequence to a one hot encoding and unknown characters in the first sequence to a zero value to create a first bit representation, and at block 504, the sequence comparison engine 210 converts value characters in the second sequence to the one hot encoding and unknown characters in the second sequence to the zero value to create a second bit representation.

Value characters are any character in the sequence that represents a specific value, while unknown characters are a character in the sequence that represents an unknown value. For example, a sequencing system 102 may generate sequence read information that includes values that indicate which of four different amino acids are detected (A, C, G, or T) for each base pair, or a value that indicates that the base pair could not be identified (N). In such an example, the A, C, G, and T characters would be value characters, and the N characters would be unknown characters.

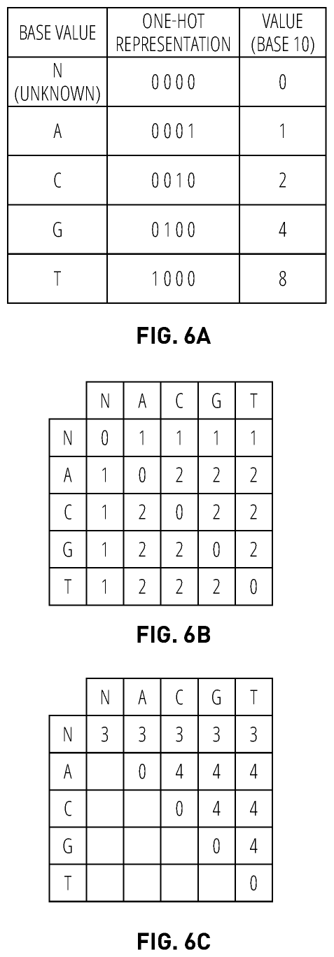

FIG. 6A illustrates a non-limiting example embodiment of a one hot encoding according to various aspects of the present disclosure. The one hot encoding illustrated in FIG. 6A is for sequence data where possible values are A, C, G, and T. Since there are four possible values, the one hot representation is four binary digits wide. A one hot representation of all zeros encodes an unknown character, while each of the value characters is represented by setting a corresponding digit in the one hot encoding to one and leaving the remainder of the digits as zero. In some embodiments, more or fewer value characters may be encoded by changing the number of digits in the one hot representation.

Returning to FIG. 5, at block 506, the sequence comparison engine 210 compares the first bit representation and the second bit representation using a bitwise XOR operation to obtain a bitwise XOR result. With the one hot representation as illustrated in FIG. 6A, the bitwise XOR operation will give rise to a bitstring where the number of ones distinguishes between matches, mismatches, and partial matches (where one of the letters is unknown). In this way, the comparison of the two sequences is converted into bitwise operations followed by bitcounting, instead of requiring N character comparisons (wherein N is the length of the sequence). The bitwise operations, in essence, allow X characters to be compared at once, where X is the bit width of the processor 202 used by the sequence comparison engine 210 divided by the number of digits in the one hot representation.

FIG. 6B illustrates the number of one bits that are present when any two characters are compared using an exclusive or (XOR, or ^) operation. As illustrated in FIG. 6A, the N (unknown character) is all zeros, while each of the value characters includes a single one in a unique position. Accordingly, an XOR of N (0000) with N (0000) yields a value that includes zero ones (0000), an XOR of N (0000) with any value character (e.g., A (0001)) yields a value that includes a single one (e.g., 0001), an XOR of any value character with a non-matching value character (e.g., A (0001) versus C (0010)) yields a value that includes two ones (e.g., 0011), and an XOR of any value character with itself (e.g., A (0001) versus A (0001)) yields a value of zero (e.g., 0000).

FIG. 6C illustrates the pairwise distance that is assigned between the characters by virtue of the one hot encoding. As shown, the distance from an N character to any other character is 3, the distance between any value character and itself is 0, and the distance from any value character to any non-matching value character is 4.

To derive the relationship between bitcounts and Hamming distance, four categories of possible pairs in corresponding characters of the first sequence (sequence “a”) and the second sequence (sequence “b”) are established:

| Category | Sequence a | Sequence b | Definition |
|---|---|---|---|
| 1 | N | N | The number of pairs of |
| | | | characters where both |
| | | | characters are unknown (“NN”) |
| 2 | N (or | [ACGT] | The number of pairs of |
| | [ACGT]) | (or N) | characters where exactly one |
| | | | corresponding character is |
| | | | unknown (“NX”) |
| 3 | [ACGT] | Same | The number of pairs having |
| | | [ACGT] | matching value characters |
| | | | (“XX”) |
| 4 | [ACGT] | Different | The number of pairs of |
| | | [ACGT] | characters having mismatched |
| | | | value characters (“XY”) |

The Hamming distance is one-fourth of the sum of the values in the distance matrix in FIG. 6C. For the sake of clarity, the sum of the values in FIG. 6C is referred to in some locations herein as a Hamming4 distance. Using the distance matrix in FIG. 6C, Hamming4 can be defined as:

Hamming4=4XY+3NX+3NN

There are two cases for calculating the Hamming distance between two sequences. Case 1 is where one sequence is known (that is, there are no Ns in one of the two sequences). Case 2 is the general case where both sequences can include unknown characters.

For Case 1, NN is zero, since only one of the sequences can have unknown characters. Accordingly, for Case 1, Hamming4 may be represented by:

Hamming4=4XY+3NX

Given the encoding in FIG. 6A, when a first sequence (sequence “a”) and a second sequence (sequence “b”) are represented in the one hot representations:

bitcount(a^b)=2XY+NX

Whenever there is an unknown nucleotide in either the first sequence or the second sequence, it will give rise to either an NX or an NN type of count. If each N is aligned with a value character, NX would be equal to the number of Ns in both sequences. Each time the unknown character is aligned with another unknown character, NX is decreased by 2:

NX=NumberofNs−2NN

In Case 1 (where NN=0), this equation can be simplified as:

NX=NumerOfNs

The results for Case 1 can then be combined as follows:

2*bitcount(a^b)=4XY+2NS

NumberOfNs=NX

2*bitcount(a^b)+NumberOfNs=4XY+3NX

Using the equation above defining Hamming4 for Case 1, the relationship between bitcount and Hamming4 can be stated as follows, where two times the number of bits in the bitwise XOR result is combined with the number of unknown characters to obtain the Hamming4 value:

2*bitcount(a^b)+NumberOfNs=Hamming4

In Case 2 (where NN is potentially>0), the number of NNs needs to be counted as well to correct the formula to obtain Hamming4. This may be done by creating two derivative bitstrings “na” and “nb” which are obtained from the first sequence and the second sequence, respectively, by assigning a bit of 1 when the character is an N and assigning a bit of 0 when the character is a value character. Accordingly, the bitwise AND (&) of na & nb will produce a value of 1 in the case of NN. In other words:

bitcount (na & nb)=NN

This may be combined with the other values as follows:

2*bitcount(a^b)=4XY+2NX

NumbeOfNs=NX+2NN

bitcount(na & nb)=NN

2*bitcount(a^b)+NumberOfNs+bitcount(na & nb)=4XY+3NX+3NN

Using the equation above defining Hamming4 for Case2, the following relationship between bitcount and Hamming4 is provided, where two times the number of bits in the bitwise XOR result is combined with the number of unknown characters and the number of NN matches to obtain the Hamming4 value:

2*bitcount(a^b)+NumberOfNs+bitcount(na & nb) Hamming4

Therefore, four times the Hamming distance can be derived using a bitwise exclusive or, a bitwise and, and bit counting operations. These few operations replace the O(n) comparisons that are used in a traditional Hamming distance calculation, and can simultaneously compare as many characters whose one hot representation will fit within the bit depth of the processor (e.g., 16 simultaneous characters that use a 4-bit one hot encoding, for a 64-bit processor).

Accordingly, returning to FIG. 5, at block 508, the sequence comparison engine 210 counts a number of bits in the bitwise XOR result (2XY) and multiplies the bitwise XOR result by two to obtain a bitcount result (4XY). At block 510, the sequence comparison engine 210 adjusts the bitcount result based on a count of unknown characters in the first sequence (+3NX). At optional block 512, the sequence comparison engine 210 adjusts the bitcount result based on a count of unknown characters in the second sequence and a count of unknown characters in the first sequence that correspond to unknown characters in the second sequence (3NN).

The actions of optional block 512 are optional because, in some embodiments, the second sequence does not include any unknown characters, in which case the adjustment does not have to be made. This could be the case in embodiments wherein, for example, the first sequence represents a sequence read from a sequencing system 102 while the second sequence represents a reference sequence from the reference sequence data store 208. In some embodiments, an indication may be provided to the procedure 500 upon calling to indicate whether the actions of optional block 512 are necessary. In some embodiments, the procedure 500 may automatically determine whether the second sequence includes any unknown characters.

At block 514, the sequence comparison engine 210 divides the adjusted bitcount result (Hamming4) by four to obtain the Hamming distance. The procedure 500 then returns the Hamming distance to its caller as a result, and the procedure 500 exits.

Empirical testing has been performed on the procedure 500, compared to traditional Hamming distance calculations. The empirical testing was performed using an AMD Ryzen 9 4900 HS CPU. The procedure 500 was found to be 120×faster than a traditional comparison of individual characters when comparing random sequences. Larger vector units for bitwise operations would be available on GPUs, newer CPUs, and custom circuitry, and would provide even greater increases in performance.

### Fictitious Example

To illustrate the calculations described above, a non-limiting example of the calculations described above is provided.

Suppose a first sequence (“sequence a”, which is “AACGNTTCC”) and a second sequence (“sequence b”, which is “ANCGNNTCT”) are provided as follows:

| | Position index | | | | | | | | |
|---|---|---|---|---|---|---|---|---|---|
| | 1 | 2 | 3 | 4 | 5 | 6 | 7 | 8 | 9 |
| Sequence | A | A | C | G | N | T | T | C | C |
| a | | | | | | | | | |
| Sequence | A | N | C | G | N | N | T | C | T |
| b | | | | | | | | | |
| Category | 3 | 2 | 3 | 3 | 1 | 2 | 3 | 3 | 4 |

In the above example, we have NN=1, NX=2, XX=5, and XY=1. Accordingly, the Hamming4 distance is:

Hamming4=4XY+3NX+3NN=(4*1)+(3*2)+(3*1)=13

In the example sequences, there are a total of 4 N characters (i.e., NumberOfNs), 1 NN comparison, and 2 NX comparisons. This aligns with the indication that NX (2)=NumberOfNs (4)−2NN (1).

The bitwise representation of sequence a using the encoding of FIG. 6A would be:
0001 0001 0010 0100 0000 1000 1000 0010 0010

The bitwise representation of sequence b using the encoding of FIG. 6A would be:
0001 0000 0010 0100 0000 0000 1000 0010 1000

Accordingly, a^b would be:
0000 0001 0000 0000 0000 1000 0000 0000 1010

Therefore, bitcount(a^b) would be 4.

Sequence na would be:
000010000

Sequence nb would be:
010011000

Accordingly, bitcount(na & nb) would be bitcount(000010000), which is 1, and the Hamming4 value is calculated as:

Hamming4=2*bitcount(a^b)+NumberOfNs+bitcount(na & nb)

Hamming4=(2*4)+4+1=13

As is shown, the bitwise operations result in a matching result for the value of Hamming4.

### EXAMPLES

### Example 1

A computer-implemented method of comparing a first set of nucleotide sequences represented by a set of first strings to a second set of nucleotide sequences represented by a set of second strings, the method comprising: receiving, by a computing device, the set of first strings representing the nucleotide sequences of the first set of nucleotide sequences; determining, by the computing device, a plurality of values representing Hamming distances between the first strings and the second strings representing the second set of nucleotide sequences by performing actions comprising, for each pairing of a first string and a second string: converting any value characters in the first string and the second string to a one hot encoding and converting any unknown characters in the first string and the second string to a zero value to create a first bit representation and a second bit representation, wherein each value character represents a nucleotide in a plurality of nucleotides; comparing the first bit representation and the second bit representation using a bitwise XOR operation to obtain a bitwise XOR result; counting a number of bits in the bitwise XOR result and multiplying the bitwise XOR result by two to obtain a bitcount result; and adjusting the bitcount result based on unknown characters in at least one of the first string and the second string to obtain the value representing the Hamming distance; and providing a result based on the determined plurality of values.

### Example 2

The method of Example 1, wherein the first set of nucleotide sequences includes one or more read sequences from a sequencing device.

### Example 3

The method of Example 1, wherein the second set of nucleotide sequences includes at least one of a sequence of interest from a reference genome; a sequence of interest from a viral insertion; a sequence of a predicted protein that is not associated with a position in the reference genome; a sequence of a predicted gene that is not associated with a position in the reference genome; and partial or complete sequences of fusion genes as a result of translocation, inversion, and deletion.

### Example 4

The method of Example 3, wherein providing the result based on the determined plurality of values includes providing an alignment of the first set of nucleotide sequences to the reference genome, the viral insertion, the predicted protein, the predicted gene, or the fusion genes.

### Example 5

The computer-implemented method of Example 1, wherein the value representing the Hamming distance between the first string and the second string is a value that is four times the Hamming distance.

### Example 6

The computer-implemented method of Example 1, wherein the first string includes at least one unknown character and the second string does not include any unknown characters, and wherein adjusting the bitcount result based on unknown characters in at least one of the first string and the second string to obtain the value representing the Hamming distance includes: counting a number of unknown characters in the first string; and adding the number of unknown characters in the first string to the bitcount result.

### Example 7

The computer-implemented method of Example 1, wherein the first string and the second string each include at least one unknown character, and wherein adjusting the bitcount result based on unknown characters in at least one of the first string and the second string to obtain the value representing the Hamming distance includes: counting a number of unknown characters in the first string and the second string to obtain a number of unknown characters; determining a number of unknown characters in the first string that are aligned with unknown characters in the second string to determine a number of matched unknown characters; and adding the number of unknown characters and the number of matched unknown characters to the bitcount result.

### Example 8

The computer-implemented method of Example 7, wherein determining the number of unknown characters in the first string that are aligned with unknown characters in the second string to determine the number of matched unknown characters includes: converting any value characters in the first string and the second string to a zero bit and converting any unknown characters in the first string and the second string to a one bit to create a first unknown character bitstring and a second unknown character bitstring; comparing the first unknown character bitstring and the second unknown character bitstring using a bitwise AND operation to obtain a bitwise AND result; and counting a number of bits in the bitwise AND result to obtain the number of matched unknown characters.

### Example 9

The computer-implemented method of Example 7, wherein the first string represents a first read sequence and the second string represents a second read sequence.

### Example 10

The computer-implemented method of Example 9, wherein providing the result includes providing a clustering of the first set of nucleotide sequences and the second set of nucleotide sequences.

### Example 11

A non-transitory computer-readable medium having computer-executable instructions stored thereon that, in response to execution by one or more processors of a computing device, cause the computing device to perform actions for comparing a first set of nucleotide sequences represented by a set of first strings to a second set of nucleotide sequences represented by a set of second strings, the actions comprising: receiving, by the computing device, the set of first strings representing the nucleotide sequences of the first set of nucleotide sequences; determining, by the computing device, a plurality of values representing Hamming distances between the first strings and the second strings representing the second set of nucleotide sequences by performing actions comprising, for each pairing of a first string and a second string: converting any value characters in the first string and the second string to a one hot encoding and converting any unknown characters in the first string and the second string to a zero value to create a first bit representation and a second bit representation, wherein each value character represents a nucleotide in a plurality of nucleotides; comparing the first bit representation and the second bit representation using a bitwise XOR operation to obtain a bitwise XOR result; counting a number of bits in the bitwise XOR result and multiplying the bitwise XOR result by two to obtain a bitcount result; and adjusting the bitcount result based on unknown characters in at least one of the first string and the second string to obtain the value representing the Hamming distance; and providing a result based on the determined plurality of values.

### Example 12

The non-transitory computer-readable medium of Example 11, wherein the first set of nucleotide sequences includes one or more read sequences from a sequencing device.

### Example 13

The non-transitory computer-readable medium of Example 11, wherein the second set of nucleotide sequences includes at least one of a sequence of interest from a reference genome; a sequence of interest from a viral insertion; a sequence of a predicted protein that is not associated with a position in the reference genome; a sequence of a predicted gene that is not associated with a position in the reference genome; and partial or complete sequences of fusion genes as a result of translocation, inversion, and deletion.

### Example 14

The non-transitory computer-readable medium of Example 13, wherein providing the result based on the determined plurality of values includes providing an alignment of the first set of nucleotide sequences to the reference genome, the viral insertion, the predicted protein, the predicted gene, or the fusion genes.

### Example 15

The non-transitory computer-readable medium of Example 11, wherein the value representing the Hamming distance between the first string and the second string is a value that is four times the Hamming distance.

### Example 16

The non-transitory computer-readable medium of Example 11, wherein the first string includes at least one unknown character and the second string does not include any unknown characters, and wherein adjusting the bitcount result based on unknown characters in at least one of the first string and the second string to obtain the value representing the Hamming distance includes: counting a number of unknown characters in the first string; and adding the number of unknown characters in the first string to the bitcount result.

### Example 17

The non-transitory computer-readable medium of Example 11, wherein the first string and the second string each include at least one unknown character, and wherein adjusting the bitcount result based on unknown characters in at least one of the first string and the second string to obtain the value representing the Hamming distance includes: counting a number of unknown characters in the first string and the second string to obtain a number of unknown characters; determining a number of unknown characters in the first string that are aligned with unknown characters in the second string to determine a number of matched unknown characters; and adding the number of unknown characters and the number of matched unknown characters to the bitcount result.

### Example 18

The non-transitory computer-readable medium of Example 17, wherein determining the number of unknown characters in the first string that are aligned with unknown characters in the second string to determine the number of matched unknown characters includes: converting any value characters in the first string and the second string to a zero bit and converting any unknown characters in the first string and the second string to a one bit to create a first unknown character bitstring and a second unknown character bitstring; comparing the first unknown character bitstring and the second unknown character bitstring using a bitwise AND operation to obtain a bitwise AND result; and counting a number of bits in the bitwise AND result to obtain the number of matched unknown characters.

### Example 19

The non-transitory computer-readable medium of Example 17, wherein the first string represents a first read sequence and the second string represents a second read sequence.

### Example 20

The non-transitory computer-readable medium of Example 19, wherein providing the result includes providing a clustering of the first set of nucleotide sequences and the second set of nucleotide sequences.

While illustrative embodiments have been illustrated and described, it will be appreciated that various changes can be made therein without departing from the spirit and scope of the invention.

The embodiments of the invention in which an exclusive property or privilege is claimed are defined as follows:

1. A computer-implemented method of comparing a first set of nucleotide sequences represented by a set of first strings to a second set of nucleotide sequences represented by a set of second strings, the method comprising:
receiving, by a computing device, the set of first strings representing the nucleotide sequences of the first set of nucleotide sequences;
determining, by the computing device, a plurality of values representing Hamming distances between the first strings and the second strings representing the second set of nucleotide sequences by performing actions comprising, for each pairing of a first string and a second string:
converting any value characters in the first string and the second string to a one hot encoding and converting any unknown characters in the first string and the second string to a zero value to create a first bit representation and a second bit representation, wherein each value character represents a nucleotide in a plurality of nucleotides;
comparing the first bit representation and the second bit representation using a bitwise XOR operation to obtain a bitwise XOR result;
counting a number of bits in the bitwise XOR result and multiplying the bitwise XOR result by two to obtain a bitcount result; and
adjusting the bitcount result based on unknown characters in at least one of the first string and the second string to obtain the value representing the Hamming distance; and
providing a result based on the determined plurality of values.

2. The method of claim 1, wherein the first set of nucleotide sequences includes one or more read sequences from a sequencing device.

3. The method of claim 1, wherein the second set of nucleotide sequences includes at least one of a sequence of interest from a reference genome; a sequence of interest from a viral insertion; a sequence of a predicted protein that is not associated with a position in the reference genome; a sequence of a predicted gene that is not associated with a position in the reference genome; and partial or complete sequences of fusion genes as a result of translocation, inversion, and deletion.

4. The method of claim 3, wherein providing the result based on the determined plurality of values includes providing an alignment of the first set of nucleotide sequences to the reference genome, the viral insertion, the predicted protein, the predicted gene, or the fusion genes.

5. The computer-implemented method of claim 1, wherein the value representing the Hamming distance between the first string and the second string is a value that is four times the Hamming distance.

6. The computer-implemented method of claim 1, wherein the first string includes at least one unknown character and the second string does not include any unknown characters, and wherein adjusting the bitcount result based on unknown characters in at least one of the first string and the second string to obtain the value representing the Hamming distance includes:
counting a number of unknown characters in the first string; and
adding the number of unknown characters in the first string to the bitcount result.

7. The computer-implemented method of claim 1, wherein the first string and the second string each include at least one unknown character, and wherein adjusting the bitcount result based on unknown characters in at least one of the first string and the second string to obtain the value representing the Hamming distance includes:
counting a number of unknown characters in the first string and the second string to obtain a number of unknown characters;
determining a number of unknown characters in the first string that are aligned with unknown characters in the second string to determine a number of matched unknown characters; and
adding the number of unknown characters and the number of matched unknown characters to the bitcount result.

8. The computer-implemented method of claim 7, wherein determining the number of unknown characters in the first string that are aligned with unknown characters in the second string to determine the number of matched unknown characters includes:
converting any value characters in the first string and the second string to a zero bit and converting any unknown characters in the first string and the second string to a one bit to create a first unknown character bitstring and a second unknown character bitstring;
comparing the first unknown character bitstring and the second unknown character bitstring using a bitwise AND operation to obtain a bitwise AND result; and
counting a number of bits in the bitwise AND result to obtain the number of matched unknown characters.

9. The computer-implemented method of claim 7, wherein the first string represents a first read sequence and the second string represents a second read sequence.

10. The computer-implemented method of claim 9, wherein providing the result includes providing a clustering of the first set of nucleotide sequences and the second set of nucleotide sequences.

11. A non-transitory computer-readable medium having computer-executable instructions stored thereon that, in response to execution by one or more processors of a computing device, cause the computing device to perform actions for comparing a first set of nucleotide sequences represented by a set of first strings to a second set of nucleotide sequences represented by a set of second strings, the actions comprising:
receiving, by the computing device, the set of first strings representing the nucleotide sequences of the first set of nucleotide sequences;
determining, by the computing device, a plurality of values representing Hamming distances between the first strings and the second strings representing the second set of nucleotide sequences by performing actions comprising, for each pairing of a first string and a second string:
converting any value characters in the first string and the second string to a one hot encoding and converting any unknown characters in the first string and the second string to a zero value to create a first bit representation and a second bit representation, wherein each value character represents a nucleotide in a plurality of nucleotides;
comparing the first bit representation and the second bit representation using a bitwise XOR operation to obtain a bitwise XOR result;
counting a number of bits in the bitwise XOR result and multiplying the bitwise XOR result by two to obtain a bitcount result; and
adjusting the bitcount result based on unknown characters in at least one of the first string and the second string to obtain the value representing the Hamming distance; and
providing a result based on the determined plurality of values.

12. The non-transitory computer-readable medium of claim 11, wherein the first set of nucleotide sequences includes one or more read sequences from a sequencing device.

13. The non-transitory computer-readable medium of claim 11, wherein the second set of nucleotide sequences includes at least one of a sequence of interest from a reference genome; a sequence of interest from a viral insertion; a sequence of a predicted protein that is not associated with a position in the reference genome; a sequence of a predicted gene that is not associated with a position in the reference genome; and partial or complete sequences of fusion genes as a result of translocation, inversion, and deletion.

14. The non-transitory computer-readable medium of claim 13, wherein providing the result based on the determined plurality of values includes providing an alignment of the first set of nucleotide sequences to the reference genome, the viral insertion, the predicted protein, the predicted gene, or the fusion genes.

15. The non-transitory computer-readable medium of claim 11, wherein the value representing the Hamming distance between the first string and the second string is a value that is four times the Hamming distance.

16. The non-transitory computer-readable medium of claim 11, wherein the first string includes at least one unknown character and the second string does not include any unknown characters, and wherein adjusting the bitcount result based on unknown characters in at least one of the first string and the second string to obtain the value representing the Hamming distance includes:
counting a number of unknown characters in the first string; and
adding the number of unknown characters in the first string to the bitcount result.

17. The non-transitory computer-readable medium of claim 11, wherein the first string and the second string each include at least one unknown character, and wherein adjusting the bitcount result based on unknown characters in at least one of the first string and the second string to obtain the value representing the Hamming distance includes:
counting a number of unknown characters in the first string and the second string to obtain a number of unknown characters;
determining a number of unknown characters in the first string that are aligned with unknown characters in the second string to determine a number of matched unknown characters; and
adding the number of unknown characters and the number of matched unknown characters to the bitcount result.

18. The non-transitory computer-readable medium of claim 17, wherein determining the number of unknown characters in the first string that are aligned with unknown characters in the second string to determine the number of matched unknown characters includes:
converting any value characters in the first string and the second string to a zero bit and converting any unknown characters in the first string and the second string to a one bit to create a first unknown character bitstring and a second unknown character bitstring;
comparing the first unknown character bitstring and the second unknown character bitstring using a bitwise AND operation to obtain a bitwise AND result; and
counting a number of bits in the bitwise AND result to obtain the number of matched unknown characters.

19. The non-transitory computer-readable medium of claim 17, wherein the first string represents a first read sequence and the second string represents a second read sequence.

20. The non-transitory computer-readable medium of claim 19, wherein providing the result includes providing a clustering of the first set of nucleotide sequences and the second set of nucleotide sequences.
